(19) United States
(12) Patent Application Publication
(10) Pub. No.: US 2019/0276416 A1
(43) Pub. Date: Sep. 12, 2019

(54) METHOD FOR PREPARING NOTA DERIVATIVE

(71) Applicant: INSTITUTE OF NUCLEAR ENERGY RESEARCH, ATOMIC ENERGY COUNCIL, EXECUTIVE YUAN, R.O.C., Taoyuan City (TW)

(72) Inventors: YU CHANG, TAOYUAN CITY (TW); CHENG-FANG HSU, TAOYUAN CITY (TW)

(21) Appl. No.: 15/912,734

(22) Filed: Mar. 6, 2018

(51) Int. Cl. C07D 255/02; C07C 303/38; C07C 303/28; C07C 311/18; C07C 309/30; C07B 51/00

(52) U.S. Cl. CPC C07D 255/02; C07C 303/38; C07B 51/00; C07C 311/18; C07C 309/30; C07C 303/28

(57) ABSTRACT

A method for preparing a NOTA derivative is revealed. The method includes a plurality of steps. First take 4-toluenesulfonyl chloride and diethylenetriamine to carry out tosylation reaction and obtain a first product. Then the first substitution reaction takes place upon addition of the first product with sodium methoxide to get the second product. Next take 4-toluenesulfonyl chloride to react with ethylene glycol for preparing a third product by tosylation reaction therebewteen. Then a coupling reaction between the third product and the second product is carried out to produce a fourth product. The second substitution reaction occurs involving the fourth product in the presence of sulfuric acid. Lastly take the reaction product and hydrochloric acid to have bonding reaction and obtain a final product. The method solves the water-absorption problem of the cyclic organic compound TACN, a NOTA derivative.

### BACKGROUND OF THE INVENTION

### Field of the Invention

The present invention relates to a method for preparing a derivative, especially to a method for preparing a NOTA derivative.

### Description of Related Art

Positron Emission Tomography (abbreviated as PET) is rapidly advanced in recent years as a powerful imaging modality that has been used to investigate cancer incidence and severity, conditions of neural systems and cardiovascular diseases.

The PET has the following uses: 1. Early cancer detection 2. Cancer staging and metastasis evaluation 3. Monitoring treatment of cancers 4. cancer recurrence detection. Many researchers have demonstrated that whole body PET scanning not only can check whether the primary lesion in patients with suspected malignancy is malignant, but also can use for early detection of metastasis and accurate staging. Thus PET plays a key role in treatment selection and planning.

According to foreign research data, the stage of 33% lung cancer patients has been changed after PET scanning and 42% patients have changed their treatment because of PET results. After treatment, PET imaging of tumor metabolism can be used in evaluation of the effectiveness of the treatment. Locally recurrence or metastasis can be detected earlier by regular PET follow up after completion of cancer treatment. Take colorectal cancer as an example, recurrence appeared in about one-third patients after the first 2 years after surgery. The sensitivity and specificity of PET is 93˜100% and 86˜100% respectively.

PET is a nuclear medicine functional imaging technique. First a tracer, positron-emitting radionuclide, is introduced into the patient's body. The tracer is concentrated in specific cells/tissues with abnormal metabolism. Then 3-dimensional images of tracer concentration within the body are then constructed by PET scanners. For the most common cancer tests, the most widely used tracer is the positron-emitting radionuclide that binds to substances essential to cell metabolism (such as glucose). Thus the tracer is taken up by normal cells in the human body. However, the research found that some malignant tumor cells have unusual high metabolic rate than the normal cells. Thus there are more tracers tapped in the tumor cells than in normal cells. Therefore early detection of malignant tumor cells can be achieved by whole-body PET scanning after injection of the tracer.

The most common PET tracer by far is fludeoxyglucose (FDG), an analogue of glucose. Thus FDG-PET is used to show glucose metabolism in cells. The half-life of FDG is relatively short (109 minutes) so that no residue is left in the body and there is no side effect on the body. Most of the malignant tumor cells have increased glucose metabolism so that more FDG is trapped therein than the normal cells and the PET image shows a focus of intense FDG uptake.

As to the radionuclides used, fluorine-18 (F-18), oxygen-15 (O-15), nitrogen-13 (N-13), and carbon-11 (C-11) are the most common. They offer easy radiolabelling of biological or drug molecules. Moreover, the Fluorine has the longest half-life among the above radionuclides-109 minutes. The half-life of F-18 allows rapid synthesis within a short period and detection in vivo. The problem caused by residual radiation dose after imaging can also be avoided due to short half-life. Thus fluorine-18 is the most widely used radionuclide now.

Recently a plurality of researchers has indicated “non-carbon-fluorine module” of fluorine-18 labelling, especially the aluminum-fluoride bond whose bond energy (670 kJ/mol) is much higher than bond energy of other metal-fluoride bonds has attracted much attention. Among the related research, a great number are associated with the connection between the 1,4,7-triazacyclononane-1,4,7-triacetic acid (NOTA) ligand and Al-18F.

However, one of the NOTA derivatives-1,4,7-triazacyclononane (TACN), a cyclic organic compound, is easy to react with moisture in air and become a sticky oily mass. This leads to difficulty in weighting. Thus there is a need to solve the moisture absorption problem of TACN for preventing the product from becoming a thick paste.

### SUMMARY OF THE INVENTION

Therefore it is a primary object of the present invention to provide a method for preparing a NOTA derivative, TACN, which solves the moisture absorption problems of TACN and further overcomes the problems of thick paste and the difficulty in weighting.

It is another object of the present invention to provide a method for preparing a NOTA derivative (TACN) in which extraction and purification are carried by simple changes in pH and differences in solubility in solvents instead of complicated purification and following extraction by using silicon columns. Thereby both reaction yield and production rate are improved.

In order to achieve the above objects, a method for preparing a NOTA derivative according to the present invention includes a plurality of steps. First take 4-toluenesulfonyl chloride and diethylenetriamine to carry out tosylation reaction and obtain a first product. Then the first substitution reaction takes place upon adding the first product with sodium methoxide to obtain a second product. Next take 4-toluenesulfonyl chloride to react with ethylene glycol for preparing a third product by tosylation reaction therebewteen. Then a coupling reaction between the third product and the second product is carried out to produce a fourth product. Take the fourth product and sulfuric acid to carry out the second substitution reaction and obtain a reaction product. Lastly take the reaction product and hydrochloric acid to have bonding reaction and obtain a final product.

The present method further includes a step of adding ether for dissolution and formation of a saturated solution after the step of taking the reaction product and hydrochloric acid to have bonding reaction.

The first product is N,N′,N″-Tris(p-toluenesulfonyl)diethylenetriamine (Ts₃DET).

The second product produced is N,N′,N″-Tris(p-toluenesulfonyl)diethylenetriamine N,N″-disodium salt (Na₂Ts₃DET).

The third product is ethylene glycol ditosylate (EGT).

The fourth product is 1,4,7-tris[(4-Methylphenyl)sulfonyl]-1,4,7-triazonane (Ts₃TACN).

The reaction product is 1,4,7-Triazacyclononane (TACN).

The final product is 1,4,7-Triazacyclononane trihydrochloride (TACN.3HCL).

In the step of running the first substitution, at least one hydrogen ion of the first product is replaced by at least sodium ion of the sodium methoxide.

At least one tosyl group of the fourth product is replaced by hydrogen ion of the sulfuric acid to get the reaction product in the step of running the second substitution.

After the step of taking the fourth product and sulfuric acid to carry out the second substitution reaction and obtain a reaction product, the present method further includes a step of placing the reaction product into an ice bath for cooling.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein:

FIG. 1 is a flow chart showing synthesis steps of an embodiment according to the present invention;

FIG. 2 is a reaction pathway of the synthesis of a NOTA derivative according to the present invention.

### DETAILED DESCRIPTION OF THE PREFERRED EMBODIMENT

In order to learn features and functions of the present invention clearly, please refer to the following embodiments, related figures and detailed descriptions.

A method for preparing a NOTA derivative is provided by the present invention in order to solve the thick paste problem of the NOTA derivative-1,4,7-triazacyclononane (TACN), a cyclic organic compound.

The chemical structure and characteristics of the compounds used and the steps of the method for preparing the NOTA derivative of the present invention are further described in the following paragraphs.

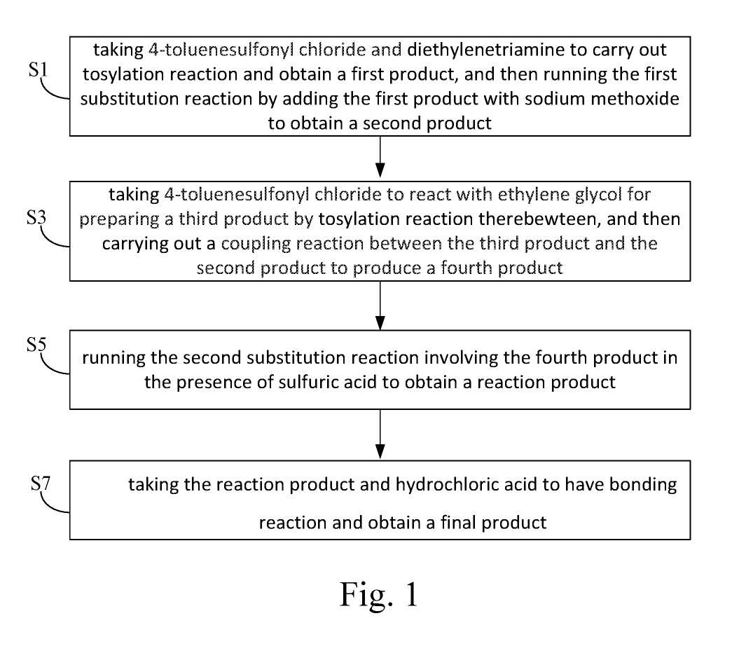

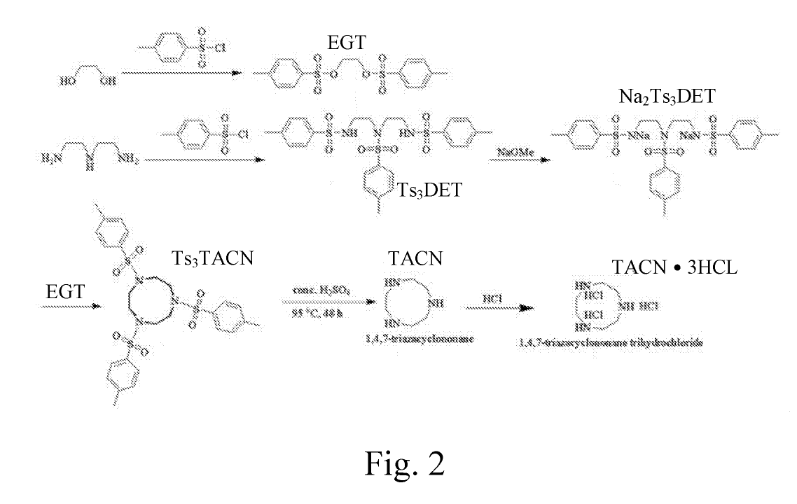

Refer to FIG. 1 and FIG. 2, a flow chart showing synthesis steps and a reaction pathway of the synthesis are revealed respectively. As shown in the figures, a method for preparing a NOTA derivative according to the present invention includes the following steps.

Step S1: Take 4-toluenesulfonyl chloride/p-toluenesulfonyl chloride and diethylenetriamine to carry out tosylation reaction and obtain a first product. Then the first substitution reaction takes place upon addition of the first product with sodium methoxide to obtain a second product.

Step S3: Take 4-toluenesulfonyl chloride to react with ethylene glycol for preparing a third product by tosylation reaction therebewteen. Then a coupling reaction between the third product and the second product is carried out to produce a fourth product.

Step S5: run the second substitution reaction involving the fourth product in the presence of sulfuric acid to obtain a reaction product; and

Step S7: take the reaction product and hydrochloric acid to have bonding reaction and obtain a final product.

As shown in the step S1, the tosylation reaction occurs under alkaline conditions. In the step of running the first substitution, at least one hydrogen ion of the first product is replaced by at least sodium ion of the sodium methoxide. The amount of the second product generated depends on the amount of the sodium methoxide being added. In order to prevent weight loss of the second product during precipitation of the second product after addition of sodium methoxide, the residual sodium methoxide is removed directly by suction instead of filtration. Thus a solid product is obtained.

The first product produced in the step S1 is N,N′,N″-Tris(p-toluenesulfonyl)diethylenetriamine (Ts₃DET) while the second product produced is N,N′,N″-Tris(p-toluenesulfonyl)diethylenetriamine N,N″-disodium salt (Na₂Ts₃DET).

Analysis data of the first product: ¹H-NMR (CDCl₃): δ 7.77 (d, C₆—H, C₁₀—H, C₁₃—H, and C₁₇—H, 4H), 7.60 (d, C₂₀—H, and C₂₄—H, 2H), 7.33 (m, C₇—H, C₉—H, C₁₄—H, C₁₆—H, C₂₁—H, and C₂₃—H, 6H), 5.20 (t, 2*N—H, 2H), 3.18 (m, C₁—H₂, C₂—H₂, C₃—H₂, and C₄—H₂, 8H), and 2.43 (s, C₁₁—H₃, C₁₈—H₃, and C₂₅—H₃, 9H); ¹³C-NMR (CDCl₃): δ 144 (C₅, C₁₂ and C₁₉), 137 (C₈ and C₁₅), 135 (C₂₂), 130 (C₇, C₉, C₁₄, C₁₆, C₂₁ and C₂₃), 127 (C₆, C₁₀, C₁₃, C₁₇, C₂₀ and C₂₄), 51 (C₁ and C₃), 43 (C₂ and C₄), and 22 (C₁₁, C₁₈ and C₂₅).

Analysis data of the second product: ¹H-NMR (CDCl₃): δ 7.77 (d, C₆—H, C₁₀—H, C₁₃—H, and C₁₇—H, 4H), 7.60 (d, C₂₀—H, and C₂₄—H, 2H), 7.33 (m, C₇—HC—H, C₁₄—H, C₁₆—H, C₂₁—H, and C₂₃—H, 6H), 5.20 (t, 2*N—H, 2H), 3.18 (m, C₁—H₂, C₂—H₂, C₃—H₂, and C₄—H₂, 8H), and 2.43 (s, C₁₁—H₃, C₁₈—H₃, and C₂₅—H₃, 9H); ¹³C-NMR (CDCl₃): δ 144 (C₅, C₁₂ and C₁₉), 137 (C₈ and C₁₅), 135 (C₂₂), 130 (C₇, 9, C₁₄, C₁₆, \vec{C}₂₁ and \vec{C}₂₃), 127 (C₆, C₁₀, C₁₃, C₁₇, C₂₀ and C₂₄), 51 (C₁ and C₃), 43 (C₂ and C₄), and 22 (C₁₁, C₁₈ and C₂₅).

As shown in the step S3, the tosylation reaction is the same as that in the step S1, taking place under alkaline conditions. The third product is ethylene di(p-toluenesulfonate)/ethylene glycol ditosylate (EGT) while the fourth product is 1,4,7-tris[(4-Methylphenyl)sulfonyl]-1,4,7-triazonane (Ts₃TACN).

Analysis data of the third product: ¹H-NMR (CDCl₃): δ 7.73 (d, C₄—H, C₈—H, C₁₁—H, and C₁₅—H, 4H), 7.34 (d, C₅—H, C₇—H, C₁₂—H, and C₁₄—H, 4H), 4.18 (s, C₁—H₂, and C₂—H₂, 4H), and 2.45 (s, C₉—H₃, and C₁₆—H₃, 6H); ¹³C-NMR (CDCl₃): δ 145 (C₃ and C₁₀), 132 (C₆ and C₁₃), 130 (C₅, C₇, C₁₂ and C₁₄), 128 (C₄, C₈, C₁₁ and C₁₅), 67 (C₁ and C₂), and 22 (C₉ and C₁₆).

Analysis data of the fourth product: ¹H-NMR (CDCl₃): δ 7.74 (d, C₈—H, C₁₂—H, C₁₅—H, C₁₉—H, C₂₂—H, and C₂₆—H, 6H), 7.27 (d, C₉—H, C₁₁—H, C₁₆—H, C₁₈—H, C₂₃—H, and C₂₅—H, 6H), 3.42 (s, C₁—H₂, C₂—H₂, C₃—H₂, C₄—H₂, C₅—H₂, and C₆—H₂, 12H), and 2.43 (s, C₁₃—H₃, C₂₀—H₃, and C₂₇—H₃, 9H); ¹³C-NMR (CDCl₃): δ 140 (C₇, C₁₄ and C₂₁), 135 (C₁₀, C₁₇ and C₂₄), 130 (C₉, C₁₁, C₁₆, C₁₈, C₂₃ and C₂₅), 127 (C₈, C₁₂, C₁₅, C₁₉, C₂₂ and C₂₆), 52 (C₁, C₂, C₃, C₄, C₅ and C₆), and 22 (C₁₃, C₂₀ and C₂₇).

Refer to the step S5 and the step S7, at least one tosyl group of the fourth product is replaced by hydrogen ion of the sulfuric acid to get the reaction product in the step of running the second substitution. After the step S5, the present invention further includes a further step of placing the reaction product into an ice bath for cooling to prevent solution from bumping and expelling from the container. The optimal temperature of the ice bath is 0° C. and the reaction time is 30 minutes. The reaction product is 1,4,7-Triazacyclononane (TACN).

In the step S7, the reaction between hydrochloric acid and water is extremely exothermic and a lot of heat is released. In order to avoid bumping and spraying of the solution, the hydrochloric acid is slowly dropped into the reaction product. After the step of taking the reaction product and hydrochloric acid to have bonding reaction, the present invention further includes a step of adding ether for dissolution and formation of a saturated solution. Thus a final product is precipitated. The mixed solution should be stirred all the time after adding the ether otherwise the yield rate is low. Then carry out vacuum filtration for 4 hours and the final product is fully dried. The solid substance of the final product is difficult to be collected from the filter paper once the final product is not dried completely and this result in weight loss of the final product, 1,4,7-Triazacyclononane trihydrochloride (TACN.3HCL).

Analysis data of the final product: ¹H-NMR (DMSO): δ 9.97 (br, 3*N+—H₂, 6H), and 3.56 (s, C₁—H₂, C₂—H, C₃—H₂, C₄—H₂, C₅—H₂, and C₆—H₂, 12H); ¹³C-NMR (DMSO): δ 42 (C₁, C₂, C₃, C₄, C₅ and C₆).

In an embodiment of the present invention, the first product is prepared by the following steps. First take 13.25 ml (95.4 mmol) triethylamine and 3.2 ml (29.8 mmol) diethylenetriamine in a 500 ml round-bottom flask and dissolve them in 133 ml dichloromethane. In an ice bath (0 degree Celsius), connect the flask to a drying tube after being added with 17.5 g (91.6 mmol) 4-toluenesulfonyl chloride and then keep stirring for 12 hours. Heat and reflux the mixture in 150 ml methanol at 80° C. for 60 minutes after vacuum concentration at 30° C. Powdered solid is collected after cooling and filtration and then is refluxed in 150 ml methanol at 80° C. for 60 minutes again. Powdered solid is collected again after cooling and filtration. All the powdered solids obtained are mixed, vacuum evaporated and condensed at 40° C. and then vacuum dried overnight to get the first product (Ts₃DET), white solid, 14.14 g (84%).

The second product is prepared by placing 14.14 g (25 mmol) first product and 200 ml ethanol in a 250 ml round-bottom flask and heating the flask until the first product is dissolved completely. Then the temperature of the solution is increased to 90° C. after addition of 10 ml 5.4M sodium methoxide (NaOMe) and then the solution is heated under reflux for 2 hours. The solution is dried by vacuum concentration at 55° C., added with 30 ml methanol, and then dried again by vacuum concentration at 45° C. Again add 30 ml methanol, and dry the solution by vacuum concentration at 45° C. The residual is vacuum dried overnight to get the second product (Na₂Ts₃DET), white solid, 15.2 g (100%).

For preparing the third product, take a 500 ml round-bottom flask and dissolve 20 ml (144 mmol) triethylamine and 27.5 g (144 mmol) 4-toluenesulfonyl chloride in 150 dichloromethane therein. Stir the solution for 15 minutes in an ice bath at 0° C. and add 3.6 ml (65 mmol) ethylene glycol. Connect the flask to a drying tube and continue stirring for 24 hours. Wash five times with 200 ml of 1N hydrochloric acid. Collect a lower organic layer, dehydrate the layer with anhydrous sodium sulfate and filtrate sodium sulfate. The organic layer is vacuum dried overnight after being vacuum concentrated at 30° C. to get the third product (EGT), white solid, 20.9 g (87%).

Take a 250 ml round-bottom flask and dissolve 10.2 g (27.6 mmol) third product in 100 ml dimethylformamide (DMF) therein for preparing a fourth product of the present invention. Also dissolve 15.2 g (25 mmol) second product in 120 ml DMF in a 250 ml two-necked round-bottomed flask and keep stirring. The third product-DMF solution is slowly dropped into the second product-DMF solution through a dropping funnel during 2 hours. Then continue stirring the mixed solution at room temperature for 2 hours. The reacted solution is vacuum concentrated and vacuum dried overnight. The concentrated residual is dissolved in 75 ml ethanol and 75 ml dichloromethane and a solid product is obtained after filtration. Next the solid product is vacuum concentrated for 4 hours and then vacuum dried overnight to get the fourth product (Ts₃TACN), white solid, 13 g (86%).

Lastly the final product of the present invention is prepared by taking a 500 ml round-bottom flask and dissolving 13 g (22 mmol) fourth product in 30 ml concentrated sulfuric acid therein. The solution is heated to 100° C. and reacted for 48 hours. After being cooled in an ice bath at 0° C. for 30 minutes, the solution is maintained in the ice bath and 150 ml 8M hydrochloric acid aqueous solution is slowly dropped into the solution of the fourth product (it takes about 3 hours). The mixed solution is poured into a 1000 ml plastic bottle. Then the plastic bottle is placed in an ice bath and 450 ml ether is slowly dropped into the mixed solution in batches. First use a glass rod to stir the mixture for 10 minutes and then use a magnetic mixer to stir the mixture vigorously for 110 minutes in the ice bath. Filter the mixture to get the solid, wash the solid with 100 ml ether and then vacuum dry the solid overnight to get the final product (TACN.3HCL), light gray solid, 1.7 g (60%).

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalent.

1. A method for preparing a NOTA derivative comprising the steps of:
taking 4-toluenesulfonyl chloride and diethylenetriamine to carry out tosylation reaction and obtain a first product, and then running the first substitution reaction by adding the first product with sodium methoxide to obtain a second product;
taking 4-toluenesulfonyl chloride to react with ethylene glycol for preparing a third product by tosylation reaction therebetween, and then carrying out a coupling reaction between the third product and the second product to produce a fourth product, the solution of the coupling reaction is vacuum concentrated and vacuum dried overnight, the concentrated residual is dissolved in 75 ml ethanol and 75 ml dichloromethane and a solid product is obtained after filtration;
running the second substitution reaction involving the fourth product in the presence of sulfuric acid to obtain a reaction product; and
taking the reaction product and hydrochloric acid to have bonding reaction and obtain a final product;
wherein the final product is 1,4,7-Triazacyclononane trihydrochloride (TACN.3HCL).

2. The method as claimed in claim 1, wherein the method further includes a step of a step of adding ether for dissolution and formation of a saturated solution to precipitate the final product after the step of taking the reaction product and hydrochloric acid to have bonding reaction.

3. The method as claimed in claim 1, wherein the first product is N,N′,N″-Tris(p-toluenesulfonyl)diethylenetriamine (Ts3DET).

4. The method as claimed in claim 1, wherein the second product produced is N,N′,N″-Tris(p-toluenesulfonyl)diethylenetriamine N,N″-disodium salt (Na2 Ts3DET).

5. The method as claimed in claim 1, wherein the third product is ethylene glycol ditosylate (EGT).

6. The method as claimed in claim 1, wherein the fourth product is 1,4,7-tris[(4-Methylphenyl)sulfonyl]-1,4,7-triazonane (Ts3 TACN).

7. The method as claimed in claim 1, wherein the reaction product is 1,4,7-Triazacyclononane (TACN).

8. (canceled)

9. The method as claimed in claim 1, wherein at least one hydrogen ion of the first product is replaced by at least sodium ion of the sodium methoxide in the step of running the first substitution reaction by adding the first product with sodium methoxide to obtain a second product.

10. The method as claimed in claim 1, wherein at least one tosyl group of the fourth product is replaced by hydrogen ion of the sulfuric acid to get the reaction product in the step of running the second substitution reaction involving the fourth product in the presence of sulfuric acid to obtain a reaction product.

11. The method as claimed in claim 1, wherein the method further includes a step of placing the reaction product into an ice bath for cooling after the step of running the second substitution reaction involving the fourth product in the presence of sulfuric acid to obtain a reaction product.
